(19) United States
(12) Patent Application Publication
(10) Pub. No.: US 2024/0008939 A1
(43) Pub. Date: Jan. 11, 2024

(54) VINE ROBOT CATHETER DEVICE

(71) Applicant: THE REGENTS OF THE UNIVERSITY OF CALIFORNIA, Oakland, CA (US)

(72) Inventors: Elliot Hawkes, Goleta, CA (US); Tania Morimoto, San Diego, CA (US)

(21) Appl. No.: 18/251,904

(22) PCT Filed: Nov. 22, 2021
(86) PCT No.: PCT/US2021/060354

§ 371 Date: May 5, 2023

Related U.S. Application Data
(60) Provisional application No. 63/125,169, filed on Dec. 14, 2020.

(51) Int. Cl. A61B 34/30

(52) U.S. Cl. CPC A61B 34/30; A61B 2034/301; A61B 2034/303; A61M 25/0119

(57) ABSTRACT

A vine robot catheter device includes an elongate outer catheter dimensioned according to a path to reach a region of a targeted anatomy, an inverted vine robot extension having its proximal end attached to the outer catheter and its distal end partially or fully inverted into itself within the outer catheter during delivery to a target site, and a fluid path in the outer catheter to apply fluid pressure to the vine robot extension to actuate the vine robot extension to evert and extend the distal end out of and beyond the outer catheter.

### PRIORITY CLAIM AND REFERENCE TO RELATED APPLICATION

The application claims priority under 35 U.S.C. § 119 and all applicable statutes and treaties from prior U.S. provisional application Ser. No. 63/125,169 which was filed Dec. 14, 2020.

### STATEMENT OF GOVERNMENT INTEREST

This invention was made with government support under grant no. 1637446 awarded by the National Science Foundation. The government has certain rights in the invention.

### FIELD

Fields of the invention include endovascular devices and robotics.

### BACKGROUND

Hawkes et al. US Patent Publication US2019/0217908, Published Jul. 18, 2019 describes a growth robot. The growth robot has a thin-walled, hollow, pressurized, compliant body that elongates the body by everting from its tip new wall material that is stored inside the body and controls the shape of the body by actively controlling the relative lengths of the wall material along opposing sides of the body. Relative lengths of the wall material along opposing sides of the body can be controlled by shortening the length of the wall material on the side facing the inside of a turn by using contracting artificial muscles mounted along the length of the body. Relative lengths of the wall material along opposing sides of the body can also be controlled by lengthening the wall material on the side facing the outside of a turn, by releasing pinches in the wall material, or by actively softening the material so that the body lengthens due to the internal pressure. Relative lengths of the wall material along opposing sides of the body can also be controlled by actively restraining the length of the wall material on the side facing the inside of a turn while allowing the wall material on the outside of the turn to lengthen.

An advancement of the growth robot technology by Hawkes et al. is provided in a soft robotic device that has an apical extension and includes fluid emission for burrowing and cleaning Such soft robots are able to burrow through sand or dirt, in a manner analogous to a plant root. The robot extends apically through eversion, while emitting fluid from the tip that fluidizes sand and soil making it possible to grow underground. That advance is disclosed in WO 2020/060858, sand in the published paper by Hawkes et al., entitled “Soft Robotic Burrowing Device with Tip-Extension and Granular Fluidization.

Hawkes et al. PCT/US2020/43932, entitled Vine Robot Tracheal Intubation Device provides an advance regarding tracheal intubation that allows Emergency Medical Technicians (EMTs) and other practitioners to have success with tracheal intubation in emergency and non-emergency scenarios. The publication discloses an everting vine robot intubation device capable of automatically and autonomously intubating the trachea and producing a lumen through which artificial ventilation may be conducted. The vine robot intubation device includes, and can consist of, a main eversion body, an intubation body, and a mouthpiece with an access port to permit fluid/pressure transfer into the bodies from a regulated pressure reservoir. The vine robot intubation device permits a hospital level success with modest medical training and experience.

Catheter devices are used to perform minimally invasive procedures, including placement of implants, such as stents and valves, and to perform surgeries. Catheter devices sometimes include an outer sheath that defines a lumen and serves as a conduit through which smaller catheters and wires may be introduced to successfully navigate the vasculature. While endovascular minimally invasive procedures are in widespread use, risks and challenges remain. The vascular anatomy is highly variable and may demonstrate excessive tortuosity and severe angulation. Such challenging vascular anatomy is often encountered in elderly patients, who are increasingly the target of endovascular procedures for the treatment of stroke, coronary artery disease, aneurysms, and other diseases.

Accessing cerebral vessels is especially difficult, as the path to cerebral vessels is challenging, including the acute turn characteristic of a Type II or Type III aortic arch. The tortuous path to small vessels requires high surgical expertise and time for the procedure. Even with great care, risks to vessel dissection are increased when accessing complex endovascular structures.

Modern catheters can include a stiffness gradient that decreases significantly towards the tip. The advantage of the less stiff tip region is that as the catheter is pushed through winding anatomy, less force is exerted on the vessel walls compared to when using a standard push-catheter, which may reduce the risk of vascular injury during endovascular procedures. See, Heit et al. “Sofia intermediate catheter and the snake technique: safety and efficacy of the sofia catheter without guidewire or microcatheter construct. Journal of neurointerventional surgery,” 10(4):401-406 (2018). Other advanced catheter devices are steerable catheters, which include pull-wires or other structures to provide the steering function. Fu et al. “Steerable catheters in minimally invasive vascular surgery,” The International Journal of Medical Robotics and Computer Assisted Surgery, 5(4):381-391 (2009). These steerable devices demand high skill to operate and suffer from increased stiffness.

Some have proposed robotic approaches to catheter steering. The Amigo™ system, for example, enables remote steering of a commercial 3 degree-of-freedom catheter using a controller that mimics the handle of a standard catheter. Khan et al. “First experience with a novel robotic remote catheter system: Amigo™ mapping trial. Journal of Interventional Cardiac Electrophysiology,” 37(2):121-129 (2013). The Magellan is another example of a robotically steered catheter, designed to bend around tight turns with fine, controlled movement. Riga et al. “Initial clinical application of a robotically steerable catheter system in endovascular aneurysm repair,” Journal of Endovascular Therapy, 16(2):149-153 (2009). These robotic platforms, along with several others, achieve their mobility and dexterity from a set of tendons, whose relative length can be changed to achieve the steering function. Kato et al. “Tendon-driven continuum robot for endoscopic surgery: Preclinical development and validation of a tension propagation model,” IEEE/ASME Transactions on Mechatronics, 20(5):2252-2263 (2014); Kutzer et al. “Design of a new cable-driven manipulator with a large open lumen: Preliminary applications in the minimally-invasive removal of osteolysis,” 2011 IEEE International Conference on Robotics and Automation, pages 2913-2920. IEEE (2011); Camarillo et al. “Mechanics modeling of tendon-driven continuum manipulators,” IEEE transactions on robotics, 24(6):1262-1273 (2008).

Magnetic navigation is another approach to catheter steering being studied but not yet in widespread clinical use and is relatively complex to use. These catheters include a magnetically responsive tip that can be controlled with an external magnetic field. Ernst et al. “Initial experience with remote catheter ablation using a novel magnetic navigation system: magnetic remote catheter ablation,” Circulation,” 109(12):1472-1475 (2004); Kim et al. “Ferromagnetic soft continuum robots. Science Robotics,” 4(33): eaax7329, 2019.

Hydrocephalus is a condition of abnormal accumulation of cerebrospinal fluid (CSF) in the brain ventricles that can lead to permanent brain damage or death if left untreated. It is estimated that 700,000 adults and 1 in 2000 infants in the United States suffer from idiopathic normal pressure hydrocephalus. The two treatments currently available to patients are CSF shunts and Endoscopic Third Ventriculostomy (ETV). ETV drains excess CSF by puncturing a hole in the floor of the third ventricle. It is often the primary treatment for hydrocephalus because it does not require implantation of a foreign body, and thereby avoids many of the long-term complications associated with CSF shunts such as infection and device malfunction. Currently, ETV cannot be performed in all groups of patients because there is no safe straight path for the surgeon to access the ventricles without high risk of damage to brain tissue or hemorrhage. T. Charles, “Complications of endoscopic neurosurgery,” Child's Nervous System, vol. 12.5, pp. 248-253 (1996). One danger of performing ETV is possible physician error in guiding the endoscope. Errors in ETV occur more frequently in a physician's early career and often stem from applying excessive normal force when sliding a flexible endoscope through the passage of the ventricle. S. P. Ambesh and R. Kumar, “Neuroendoscopic procedures: Anesthetic considerations for a growing trend: a review,” Journal of Neurosurgical Anesthesiology, vol. 12, no. 3, pp. 262-270 (2000).

In less sensitive parts of the body, increased access into constrained environments could enable interventions beyond the current limits of controllable endoscopes and catheters. Stones lodged in the calyces of the kidney are difficult to treat as standard catheters are unable to reach them. This leaves either percutaneous nephrolithotomy (passing a needle into the pelvic region) or laparoscopic procedures as more invasive procedure. Conventional endoscopes are also unable to access the small intestine due to discomfort and lack of flexibility. Iddan et al. “Wireless capsule endoscopy,” Nature, vol. 405, no. 6785, p. 417 (2000). This presents a challenge for simple tasks such as imaging or collecting tissue samples without performing surgery. Typical ventriculoscopes used in neuroendo-scopic procedures have an active tip deflection of 160 degrees down and 100 degrees up. G. Cinalli, “Endoscopic third ventriculostomy,” Pediatric Hydro-cephalus, pp. 361-388 (2005). Endoscopes for ureteroscopy, the diagnosis and treatment of intrarenal kidney stones, can each tip deflections up to 270 degrees. This flexibility is often used when nearby the target to aid with visualization. However, in both cases, the deflection is greatly reduced introducing a tool in the working channel. Pasqui et al. “Impact on active scope deflection and irrigation flow of all endoscopic working tools during flexible ureteroscopy,” European Urology, vol. 45, pp. 58-64 (2004). Serious injury can occur when the endoscope does not bend fully to the intended trajectory.

### SUMMARY OF THE INVENTION

A preferred embodiment provides a vine robot catheter device that includes an elongate outer catheter dimensioned according to a lumen path to reach a region of a targeted anatomy, an inverted vine robot extension having its proximal end attached to the outer catheter and its distal end partially or fully inverted into itself within the outer catheter during delivery to a target site, and a fluid path in the outer catheter to apply fluid pressure to the vine robot extension to actuate the vine robot extension to evert and extend the distal end out of and beyond the outer catheter.

### BRIEF DESCRIPTION OF THE DRAWINGS

FIGS. 1A-1F are schematic views that illustrate making and completion of a preferred vine robot catheter device;

FIG. 2 illustrates a preferred catheter system with a vine robot catheter extension;

FIG. 3 is a simplified view illustrating the vine robot catheter extension of FIG. 2; and

FIG. 4 is a simplified view illustrating a vine robot catheter extension system with additional markers;

### DESCRIPTION OF THE PREFERRED EMBODIMENTS

In the following description, proximal indicates the direction toward a surgeon operating a device and distal indicates the direction away from a surgeon. A distal end is most distant from a surgeon and a proximal end is closest to a surgeon.

Preferred embodiment vine robot catheter devices include a catheter with a soft, extendable extension that grows beyond another tube, such as a standard catheter tube while that catheter tube remains stationary. The soft, extendable extension is formed as a hollow tubular structure and is attached in a sealed fashion to another catheter tube. Fluid pressure (by a gas or a liquid) provided through the delivery device causes the vine robot extension to extend while the rest of the delivery device remains stationary. This limits shear forces with the environment, easing movement of a body's extension through a constrained space. The vine robot extension is very gentle on the structure that it expands into, with minimal to no sliding movement as the vine robot extension everts and its distal end moves forward (distally) until reaching full eversion and extension. Compared to standard catheters, the vine robot catheter extension can reduce normal forces applied by one hundred-fold when navigating a 30-degree bend in anatomy.

A preferred embodiment vine robot catheter extension consists of a thin-walled, hollow biocompatible plastic or fabric tube that can be folded inside of itself, or inverted, and pressured to evert and extend, or grow, into a biological structure. Suitable plastic or fabric tubes include thermoplastic polyurethane (tpu), dyneema, PTFE, or ripstop nylon fabric with silicone or urethane coating. Generally, the material must be capable of eversion through fluid pressure, should be softer than the body lumen that it is used with, and should be biocompatible or capable of being coated with a biocompatible material. The vine robot catheter extension has diameter generally matching and preferably slightly smaller than that of the biological structure, and has a distal end that is smaller diameter to allow for blood flow around the distal end of the vine robot catheter extension. The vine robot catheter extension is attached via a mount, mechanical interface (such as threaded connection), welding, adhesive or other adhesion to the distal end of a standard catheter. The attachment is preferably fluid tight so that fluid pressure provided through the delivery device can efficiently cause the vine robot extension to evert.

During assembly of a vine robot catheter device of the invention, the inversion process can be accomplished by tucking one end of the vine robot catheter extension into the standard catheter using a rigid ramrod or by pulling a string secured to the end. During use, actuation of the vine robot catheter device occurs via applied internal pressure through the standard catheter portion to which it is attached, e.g., a catheter outer sheath or catheter inner tube in a system that has additional components within the catheter inner tube, which causes the inverted material to extend out of the extension by eversion and grow with its distal end beyond the standard catheter component that its proximal end is attached to. In addition to gentle extension and advancement, the pressure applied by the vine robot extension can be controlled. After extension, additional fluid pressure can be supplied to apply pressure externally to a biological structure, in a manner analogous to a balloon. An example material for a preferred vine robot catheter extension is a low-density polyethylene (LDPE) sheet, with a preferred thickness on the order of 10±5 microns, rolled into a tube. The thinnest possible material is preferable to have the smallest bending stiffness to minimize resistance to eversion, improving apical extension and the accuracy of pre-formed angles, with the constraint of holding the required pressure. Standard guiding catheters used for ETV or kidney stone removal have a maximum diameter of 4 mm (12 French), with smaller diameters desired. Soft vine robot catheter extensions can be formed to the specified diameter and varying lengths using a heat sealer or other adhesive method. Heat can also be used to impart a pre-bent section. Applying heat to a particular section can relax internal stress in the plastic and allow it to re-form to a desired shape. Another technique for forming a desired predetermined shape is to place the vine robot into a curved mold and apply an additional piece of material or adhesive along the inside of the curve to hold the desired shape.

Preferred embodiment vine robot catheter devices can access portions of the anatomy that are difficult or impossible to reach with standard catheters or endoscopes. Preferred vine robot catheter devices have promise in applications such as accessing blood vessels, brain ventricles, the calyces of the kidney and the small intestine via minimally invasive catheter procedures. Preferred vine robots can be tapered, as well. Preferred vine robot catheter devices can be sized and shaped, for example, to enter the colon, esophagus, and particular regions of vasculature. The proximal end of the vine catheter may have a diameter between 1 and 10 mm, preferably between 3 and 7 mm, particularly preferably 5 mm. The distal end may be the same diameter, or may be tapered, e.g., by 3 mm or 2 mm, or more preferably 1.5 mm or 0.5 mm. An example experimental vine robot catheter device has a 5 mm at proximal end to fit over a 15 Fr vascular sheath, and it is 3.5 mm at its distal end, where it is attached to a 6 Fr diagnostic angiographic catheter.

A preferred embodiment vine robot catheter device includes a catheter with inner and outer elongated tubes (e.g., an outer sheath and inner catheter tube) sized to enter vascular structures and a vine robot extension that can be actuated to evert and gently extend forward into the vascular structure after being delivered by the outer tubes. In a preferred device, the distal end of the vine robot extension is attached to the distal end of the inner catheter tube and the proximal end of the vine robot extension is attached to the distal end of the outer sheath. The attachment is preferably fluid tight so that fluid pressure provided through the delivery device can cause the vine robot extension to extend. In referring to the distal and proximal ends of the vine robot extension, those ends are a reference to its state after delivery and when it is everted and extended. The total length and diameter can be predetermined to match different physiology, e.g., by patient size, age, or measured physical characteristics. Typical outer sheaths can be a meter or more in length and can have diameters in the range of 3 Fr to about 25 Fr.

A preferred vine robot catheter device includes an elongate outer catheter dimensioned according to a lumen path to reach a region of a targeted anatomy, an inverted vine robot extension having its proximal end attached to the outer catheter and its distal end partially or fully inverted into itself within the outer catheter during delivery to a target site, and a fluid path in the outer catheter to apply fluid pressure to the vine robot extension to actuate the vine robot extension to evert and extend the distal end out of and beyond the outer catheter.

The device can include an elongate inner element within a lumen of the outer catheter, the inner element being attached to the distal end of the inverted vine robot extension. The inner element can be an inner catheter tube defining a lumen.

A guide wire can be within the inner catheter tube that is extendable to guide a path of growth of the vine robot extension to follow an anatomical path. In addition, any conventional tool (microcatheter, stent retriever, etc) can be deployed through the inner catheter once the vine robot extension has everted.

The vine robot catheter device can have one or more pre-bent (pre-curved) sections in the vine robot extension that forms a predetermined shape during eversion to follow an anatomical path.

The vine robot catheter extension can be dimensioned for the upper gastrointestinal tract, in particular esophagus, stomach, and/or small intestine, and preferably has an outer diameter in the range of 5 mm to 25 mm.

The vine robot catheter extension can be dimensioned for the lower gastrointestinal tract, in particular colon and/or rectum, and preferably has an outer diameter in the range of 5 mm to 25 mm.

The vine robot catheter extension can be dimensioned for the biliopancreatic tract, in particular common bile duct, intrahepatic bile ducts, cystic duct, and/or Wirsung canal, and preferably has an outer diameter in the range of 1 mm to 5 mm.

The vine robot catheter extension can be dimensioned for the urinary tract, in particular calyces of the kidney, ureter, bladder, and/or urethra, and preferably has an outer diameter in the range of 1 to 4 mm.

The vine robot catheter extension can be dimensioned for the respiratory tract, in particular trachea, bronchi, and/or bronchioles and preferably has an outer diameter in the range of 1 to 10 mm.

The vine robot catheter extension can be dimensioned for the uterine cavity and preferably has an outer diameter in the range of 1 to 5 mm.

The vine robot catheter extension can be dimensioned for blood vessels and preferably has an outer diameter in the range of 1 to 10 mm.

The vine robot extension can be made of a material selected from the group of plastics and fabrics, preferably thermoplastic polyurethane (tpu), dyneema, PTFE, or ripstop nylon fabric with silicone or urethane coating.

A vine robot catheter system includes a vine robot catheter device of the invention and a fluid actuation system and a pressure sensor providing real-time feedback such that the fluid actuation system can control the rate of eversion of the vine robot catheter extension.

The system can include a linear actuator to apply fluid pressure and a proportional controller that updates the speed of the linear actuator based on the difference between the current pressure and the desired pressure, where a larger pressure difference results in a higher speed of eversion.

Preferred embodiments of the invention will now be discussed with respect to the drawings and experiments, which will be understood by artisans to illustrate broader aspects of the invention in view of the general knowledge in the art and the description that follows. As an example, the vine robot catheter device could carry a camera. A camera could replace the inner catheter and be pulled out during eversion. A camera could also be placed through the inner catheter after full eversion has taken place (like any other tool). A camera could be mounted at the distal tip of the vine robot and “ride” along at the tip, being pushed forward by eversion. Other tools could also be similarly mounted, e.g., a laser ablation tool.

An experimental device demonstrates aspects of the invention and will be discussed next. Techniques for fabrication of everting vine robot are disclosed in Hawkes et al. US Patent Publication US2019/0217908, and those techniques can be used.

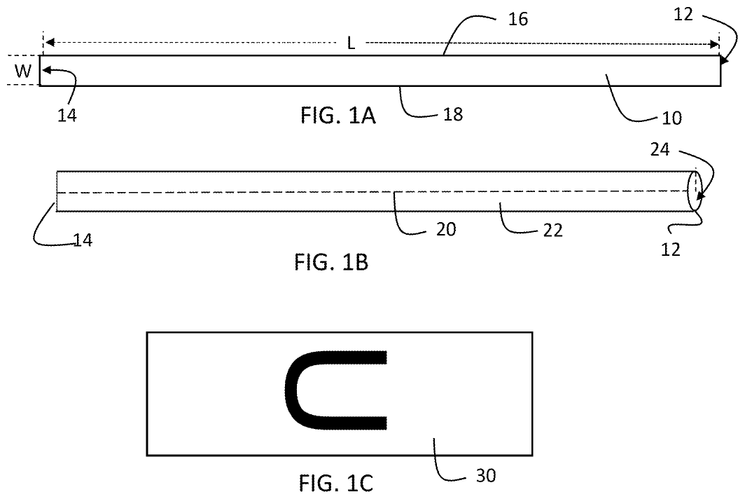

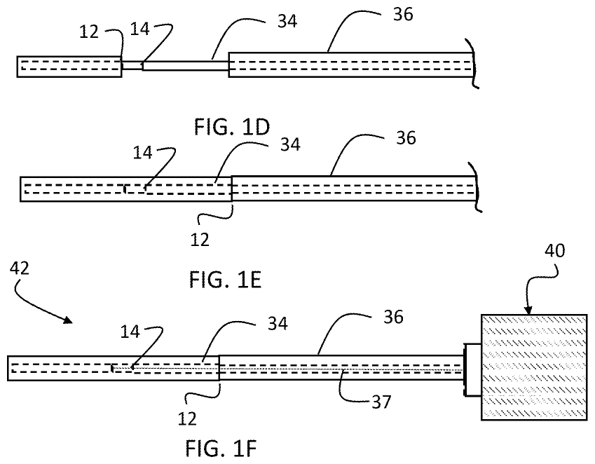

Fabrication of the experimental device starts with FIG. 1A cutting a urethane-coated ripstop nylon into the desired shape of the everting vine extension. The cut shown in FIG. 1A is straight, forming an elongate nylon rectangle 10 having the same width through its length, with the width being much smaller than the length, such that a proximal end 12 and a distal end 14 have the same width “W”. A tapered version includes “W” at the distal end 14 being smaller than “W” at the proximal end 12. FIG. 1B illustrates the result of sealing one edge 16 to the other edge 18 in a lap joint 20 to form a long, narrow tube vine robot catheter extension 22 that defines an open lumen 24 throughout its length. An optional step is to include pre-defined shape, and this is achieved, for example, in FIG. 1C with a U-shaped 3D-printed mold 30 that can be used to form the pre-bent shape in the vine robot catheter extension 22.

In FIG. 1D, the vine robot catheter extension 22 is then inverted (distal end 14 is pulled inward through lumen 24 and through proximal end 12) and the distal end 14 is attached to a distal end of an inner catheter tube 34 that extends from a lumen of an outer catheter sheath 36. A guide wire 37 is also shown within the inner catheter tube 34. In FIG. 1E, the proximal end 12 of the vine robot catheter extension 22 is attached to a distal end of the outer sheath 36.

Attachment to a conventional handle 40 having at least a fluid port for supplying fluid to the inner tube 24 creates the FIG. 1F vine robot catheter device 42. A typical handle 40 will have proximal connections for introduction of fluid to achieve everting operation of the vine robot catheter device 42 and operation of the inner catheter tube 34 and outer sheath 36, and connections such as Luer-Lock style connectors. The handle 40 can also include conventional features for advancing the catheter system into a body lumen, including joint and relative movement between the outer sheath 36 and the inner tube 34. An example operation is conventional to advance the inner tube 34 and outer sheath until the activation via fluid pressure of the vine robot catheter extension 22.

Because the vine robot catheter extension 22 can grow via eversion from a distal end of the outer sheath 26, rather than being pushed, it does not require the stiffness of even the softest catheter tubes in commercial use. Instead, it can be fabricated, for example, using a 40-denier urethane-coated ripstop nylon (Rockywoods Fabrics) that is 0.1 mm thick and nearly inextensible. The fabric can be cut into the desired shape using a stencil, preferably designed to enable a gradual taper in diameter. In the example experimental device, a diameter taper was achieved with approximately 3.5 mm at the distal end and approximately 5 mm at the proximal end. A fast-cure polyurethane glue, Marine Adhesive Sealant Fast Cure 5200 (3M), was used to seal one edge of the fabric to the other in a lap joint in order to form a long, narrow tube. Other medical adhesives and/or heat sealing can also be used. In the experimental device, after curing for approximately 24 hours, a U-shaped, 3D-printed mold (FIG. 1C) with a radius of curvature of 20 mm and a diameter of 3.7-3.9 mm depending on the resulting tube diameter was placed inside the nylon tube. A thin strip of the same ripstop nylon is then glued along the inside of the U-shaped bend, in order to create a permanent pre-bent shape with the desired curvature. Once cured, the mold is removed. As mentioned above, this pre-bent shape is optional. Extrusion and other techniques used for manufacturing thin tubes with lumens can also be used. Techniques that provide a thin wall, low friction vine robot tube that is nearly inextensible, and withstand eversion pressures can be used.

The hollow, pre-curved nylon tube that forms the vine robot extension 22 was then integrated with a catheter system of FIG. 1F. First, the vine robot catheter extension is inverted inside out by pulling the 3.5 mm distal end towards the 5 mm proximal end. Then a flexible 6-Fr diagnostic angiographic catheter (MicroVention) is fed through the sheath, and its end is attached to the inverted vine robot catheter 22 extension at the 3.5 mm distal end. Next, the catheter is pulled so the distal end of the vine robot catheter extension will slide into the 15 Fr vascular sheath and the proximal end, which has been designed to fit snugly around the sheath, will mate with the sheath distal end. In other catheter systems, there may be inner and outer catheter tubes inside an additional sheath. In such systems, the vine robot extension is attached to the inner and outer tubes. Other variations for other systems will be apparent to artisans. The key is that the vine robot extension be attached for distal delivery to the catheter system in a manner that allows actuation via fluid to evert and distally grow the vine robot extension to extend from its proximal point of connection to the catheter system.

An alternative to the pre-bent shape is to include a guide wire that the soft vine robot extension can follow around a complex structure, such as the aortic arch. The wire can be guided past the arch using standard practice, and the vine robot extension 22 follows the wire in a manner similar to how an inner catheter tube can follow a wire.

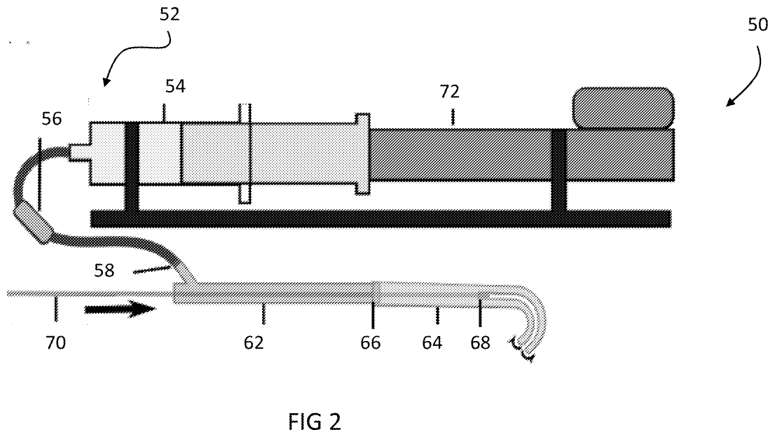

FIG. 2 show a preferred experimental vine robot catheter system 50 of the invention with its fluid actuation system 52, having a pressured syringe 54 and pressure sensor 56 with tubing connected to a fluid port 58. A vine robot catheter device 60 in FIG. 2A is consistent with the device in FIGS. 1A-1F and includes an elongate outer catheter 62 dimensioned according to a lumen path to be inserted in the region of a targeted anatomy, an inverted vine robot extension 64 having its proximal end 66 attached to the outer catheter 62 and its distal end 68 partially or fully inverted into itself within the outer catheter 62 during delivery to a target site and connected to a distal end of an inner tube 70, and a fluid path in the outer catheter to apply fluid pressure to vine robot extension 64 to actuate the vine robot extension 64 to evert and extend the distal end 68 out of and beyond the outer catheter 62. Fluid pressure can be controlled via a linear actuator 72. The experimental system was constructed as shown in FIG. 2, and included a saline-filled syringe whose tip connected to the vine robot catheter device, and whose plunger is connected to a linear actuator. The syringe and linear actuator were rigidly mounted, such that as the linear actuator pushes the plunger forward, saline is pushed into vine robot catheter device, increasing the internal pressure of the vine robot catheter extension. A pressure sensor provided real-time feedback on the pressure inside the vine robot catheter device. To grow, a proportional controller updated the speed of the linear actuator based on the difference between the current pressure and the desired pressure, where a larger pressure difference results in a higher speed. When the growing force at the distal end of the vine robot catheter extension due to the internal pressure is balanced by the tension on the inner catheter, an equilibrium is reached and no further growth occurs.

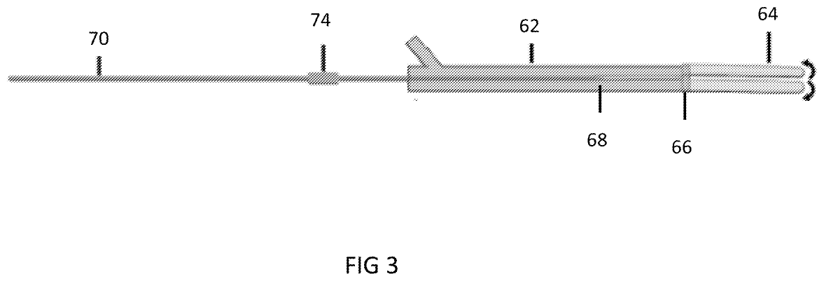

During a preferred control operation, an endovascular surgeon feeds the inner catheter forward into the outer sheath using typical controls and monitoring. With reference to the simplified drawing in FIG. 3 that is labeled consistently with FIG. 2, because the inner catheter 70 is connected to the distal end 68 of the vine robot catheter extension 64, feeding the inner tube 70 forward produces slack in the growing component, temporarily leading to a decrease in the tension of vine material of the VINE (vine robot catheter extension) catheter 64. This decrease in tension results in a net force on the VINE catheter in the direction of growth, since the controller is designed to maintain a constant pressure inside the VINE catheter. If there is a pre-bent section, to notify users of when to expect the pre-bend to emerge from the end of the VINE catheter, a marker 74 can be attached to the inner catheter, such that this marker reaches the base of the outer catheter 62 just as the pre-bend is emerging. The marker 74 can be designed to prevent further growth once it reaches this point by mechanical interference with the outer catheter, since the inner tube 70 cannot be fed any further. The users are therefore required to remove the marker 74 and orient the vine robot catheter 64 by rotating the base of the outer sheath 62, before continuing to grow. After the target location has been reached and any procedures performed, the vine robot catheter extension 64 is depressurized and pulled from the base to retract it similar to a standard catheter. The experimental vine robot catheter device and system were tested with benchtop tests and ex vivo experiments that simulated clinical use.

Benchtop Testing.

Safety—The pressure control system also serves to limit the maximum applied stress that can be induced by the extension of the robot to the surrounding anatomy. Unlike traditional push-catheters, which are relatively stiff structures pushed from the base and capable of causing dissection or other injury of the arterial vessels, the maximum stresses applied by the vine robot catheter extension are inherently limited by the internal pressure in the soft structure to be well below a dangerous level. Arterial perforation forces have been measured at around 2 N for several 4-French catheters resulting in an applied stress of around 1440 kPa. In contrast, the maximum applied stress of a 4.9 mm diameter vine robot catheter extension, when grown through a hollow channel 12.5 mm in diameter, was measured to be 165.9±8.3 kPa on average, when grown with a pressure of 200 kPa, and 234.3±25.9 kPa on average, when grown with a pressure of 300 kPa. These measured stress levels are approximately 6-8 times smaller than those that have been shown to cause arterial perforation using standard catheters. Even in the case that the full internal pressure was applied to the arterial wall, the stress level would be roughly 5 times less than the perforation stress.

Pre-Bent Section Robustness—The experimental vine robot catheter extension had a “U”-shape with a radius of curvature of 20 mm, selected to enable navigation around a range of possible angles found at the base of the aortic arch. The robustness of the device to different angles was tested. The standard semi-rigid catheter is only able to traverse curves as sharp as 90° while the vine robot catheter extension can navigate through channels with angles ranging from 180° (straight line to distal direction) to 0° (complete u-turn back toward proximal direction). To navigate these different paths, the vine robot catheter extension is grown until the pre-bend begins to emerge, at which point the base of the vine robot catheter extension is rotated to align the tip of the pre-bend in the desired direction. The vine robot catheter extension can then continue growing, relying on interaction with the constrained environment to continue along the desired path. The pre-curved vine robot catheter extension was grown through an acrylic model with dimensions similar to those of the vasculature. Compared to a straight vine robot catheter, which would always grow down a straight path if it exists and is not guided (such as by a wire discussed above), the pre-curved VINE catheter extension was able to successfully grow down angled paths angles ranging from 180° (straight line toward distal direction) to 0° (complete u-turn back toward proximal direction). As a comparison, a standard push-catheter (5-French Berenstein Catheter (Cordis, Santa Clara, CA)) was similarly navigated through the same set of acrylic models. Unlike the present catheter extension, which was able to successfully grow through all five paths, the push-catheter was only able to traverse the 180° (straight) path, and deflections in the distal direction along a 135° path, and a 90° path. Both paths back toward proximal of a 45° and 0° paths proved to have too tight a curvature for the standard push-catheter to be able to hook and translate successfully without the use of a guidewire or other tools. The difficulty in navigating standard push-catheters through these acute turns can be attributed to their method of movement. After they are hooked around a curve, the entire catheter body must translate forward in order to advance the extension towards the target, meaning that a bend in the catheter will no longer match the curve in the vasculature. As such, pushing forward might not result in forward motion around the curve. Depending on the curvature, this could be difficult to impossible without the use of additional instruments. In contrast, the vine robot catheter extension with a pre-bent section can be grown with a bend in its body that roughly matches a curve in the vasculature, and this bend can then remain stationary with respect to the environment as the extension continues to advance towards the target. Accordingly, the curves in the vasculature remain matched by the bends in the body as the end of the vine robot catheter extension continues to advance.

Ex Vivo Navigation Testing Under Fluoroscopy

Novice users and expert surgeons participated in a user study designed to compare the performance of the vine robot catheter device and a standard set of manual tools in an ex-vivo model under fluoroscopy. The set of standard instruments included a French vascular sheath, a 5-French Simmons 2 catheter, and a guidewire. The goal of the task was to navigate a catheter to a target site in the distal right common carotid artery of the model.

Experiment Procedure—Each participant first read a step-by-step explanation and watched a video of how to use both the standard catheter and the vine robot catheter device. The participants had 5 minutes to practice using each catheter device before trying the task under fluoroscopy. The novice users did their final test session after an additional 30 minutes of training—up to 15 minutes for the standard method and up to 15 minutes for the vine robot catheter method—in order to become more familiar with the devices and improve their techniques. Approval for testing the vine robot catheter under fluoroscopy was given by the University of California San Diego Institutional Review Board. All participants took an online radiation safety training course and wore protective lead aprons and a dosimetry badge. A protective shield was also used when possible. Participants were also told that they could choose to end the study at any time.

Experiment Setup—A silicone flow model of the aortic arch was used for the ex-vivo navigation task. CTA images from a rotational angiogram were used to reconstruct a 3D model of the anatomy in the area of interest, by first segmenting the 2D axial slices and then reconstructing a 3D volume from this stack of 2D segmentations. This 3D DICOM data was then used to generate a patient-specific model that was 3D printed using an FDM (Fused Deposition Modeling) printer. The model was then dip-coated using Silicone, and once cured, the model was removed. This flow model was attached to an acrylic box, fabricated to catch any liquid that may leak from the model during the experiments. The model was filled completely with water, and air bubbles were eliminated. The actuation system was clamped to the X-ray bed, and the syringe was filled with water spiked with contrast in approximately a 1:1 ratio. An external monitor was used to visualize the X-ray images in real time, and participants were shown on the monitor their target path and end location. All measurements of radiation dosage and elapsed time were recorded from the monitor.

Experimental Results—All participants were able to reach the target site beyond the arch by growing the vine robot catheter device, however none—novice nor expert—was able to complete the task using the standard set of tools. It should be noted that while this participant was able to gain initial access into the innominate artery, they were unable to further advance the catheter. We found that the experts were able to navigate into the right common carotid artery much faster using the standard tools than the novices, two out of three of whom were unable to select this branch at all. On average, it took the experts 0.67±0.05 min to navigate into the right common carotid artery and advance the guidewire towards the target, while the one novice, successful at this portion of the task, completed it in 8.1 min. On average, for the vine robot catheter device it took novices 4.22±1.90 minutes and experts 3.75±0.90 minutes, for a difference of only 0.47 minutes between the two groups. The vine robot catheter was significantly faster for all study participants compared to the actual procedure with standard tools, which took approximately 35 minutes for an expert neurointerventionalist. The potential for faster procedures has many benefits and can reduce surgical risks. One benefit is lower radiation exposure for both surgeons and patients, particularly for navigating highly tortuous anatomy.

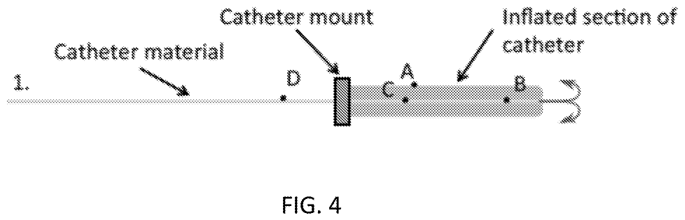

FIG. 4 shows another catheter device in which a mount mounts the vine robot catheter extension to the distal end of a standard catheter on an outer tube or a sheath, and an inner catheter of catheter material is pulled out from the catheter mount upon inflation/pressurization and eversion growth of the extension, or the inflated section of the catheter device. Markers A, B, C, and D provide information to a practitioner about the insertion of the standard catheter, the eversion growth of the vine robot extension, and the associated movement of the inner catheter as it is pulled by the robot extension. The additional markers are useful if there are multiple time-steps of eversion, enabling a practitioner to see where each part of the material ends up after each time step of eversion.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A vine robot catheter device, comprising an elongate outer catheter dimensioned according to a lumen path to reach a region of a targeted anatomy, an inverted vine robot extension having its proximal end attached to the outer catheter and its distal end partially or fully inverted into itself within the outer catheter during delivery to a target site, and a fluid path in the outer catheter to apply fluid pressure to the vine robot extension to actuate the vine robot extension to evert and extend the distal end out of and beyond the outer catheter.

2. The vine robot catheter device of claim 1, comprising an elongate inner element within a lumen of the outer catheter, the inner element being attached to the distal end of the inverted vine robot extension.

3. The vine robot catheter device of claim 2, wherein the inner element comprises an inner catheter tube defining a lumen.

4. The vine robot catheter device of claim 3, comprising a guide wire within the inner catheter tube that is extendable to guide a path of growth of the vine robot extension to follow an anatomical path.

5. The vine robot catheter device of claim 1, comprising a pre-bent section in the vine robot extension that forms a predetermined shape during eversion to follow an anatomical path

6. The catheter device of claim 1, wherein the vine robot catheter extension is dimensioned for the upper gastrointestinal tract, in particular esophagus, stomach, and/or small intestine, and preferably has an outer diameter in the range of 5 mm to 25 mm.

7. The catheter device of claim 1, wherein the vine robot catheter extension is dimensioned for the lower gastrointestinal tract, in particular colon and/or rectum, and preferably has an outer diameter in the range of 5 mm to 25 mm.

8. The catheter device of claim 1, wherein the vine robot catheter extension is dimensioned for the biliopancreatic tract, in particular common bile duct, intrahepatic bile ducts, cystic duct, and/or Wirsung canal, and preferably has an outer diameter in the range of 1 mm to 5 mm.

9. The catheter device of claim 1, wherein the vine robot catheter extension is dimensioned for the urinary tract, in particular calyces of the kidney, ureter, bladder, and/or urethra, and preferably has an outer diameter in the range of 1 to 4 mm.

10. The catheter device of claim 1, wherein the vine robot catheter extension is dimensioned for the respiratory tract, in particular trachea, bronchi, and/or bronchioles and preferably has an outer diameter in the range of 1 to 10 mm.

11. The catheter device of claim 1, wherein the vine robot catheter extension is dimensioned for the uterine cavity and preferably has an outer diameter in the range of 1 to 5 mm.

12. The catheter device of claim 1, wherein the vine robot catheter extension is dimensioned for the vessels and preferably has an outer diameter in the range of 1 to 10 mm.

13. The catheter device of claim 1, wherein the vine robot extension is made of a material selected from the group of plastics and fabrics, preferably thermoplastic polyurethane (tpu), dyneema, PTFE, or ripstop nylon fabric with silicone or urethane coating.

14. A vine robot catheter system including a vine robot catheter device of claim 1, comprising a fluid actuation system and a pressure sensor providing real-time feedback such that the fluid actuation system can control the rate of eversion of the vine robot catheter extension.

15. The vine robot catheter system of claim 14, comprising a linear actuator to apply fluid pressure and a proportional controller that updates the speed of the linear actuator based on the difference between the current pressure and the desired pressure, where a larger pressure difference results in a higher speed of eversion.

16. A method for operating a vine robot catheter system, the method comprising:
distally advancing a catheter comprising an outer sheath and an inner tube into a lumen a first distance within the lumen;
with fluid pressure, activating a vine robot catheter extension having its distal end inverted and connected to a distal end of the inner tube and its proximal end connected to a distal end of the outer sheath to evert and grow and thereby pull the inner tube distally; and
monitoring fluid pressure to maintain pressure within the vine robot catheter extension.

17. The method according to claim 16, further comprising monitoring a marker on the inner tube and pausing growth when the marker advances a predetermined distal amount.

18. The method of claim 16, wherein the vine robot catheter system comprises a pre-bent section, and further comprising rotating the outer sheath after pausing the growth and continuing growth after the rotating.
